(19) United States
(12) Patent Application Publication
(10) Pub. No.: US 2023/0331775 A1
(43) Pub. Date: Oct. 19, 2023

(54) METHOD FOR RECOVERING A PROTEIN FROM A FERMENTATION BROTH COMPRISING A HIGH DEGREE OF LYSED CELLS

(71) Applicant: BASF SE, Ludwigshafen (DE)

(72) Inventors: Sebastian Schoof, Ludwigshafen (DE); Ronja Muenkel, Ludwigshafen (DE)

(21) Appl. No.: 18/026,860

(22) PCT Filed: Sep. 21, 2021
(86) PCT No.: PCT/EP2021/075929

§ 371 Date: Mar. 17, 2023

(30) Foreign Application Priority Data
Sep. 22, 2020 (EP) 20197549.7

(51) Int. Cl. C07K 1/34; C12N 1/20

(52) U.S. Cl. CPC C07K 1/34; C12N 1/205; C12R 2001/10

(57) ABSTRACT

The present invention relates to a method for recovering a protein of interest from a bacterial fermentation broth, wherein the bacterial cells in the fermentation broth exhibit a high degree of lysis, comprising the steps of adding to the fermentation broth comprising the protein of interest at least one flocculant in an amount of 0.5 to 50 g/l of the fermentation broth; separating the protein of interest from at least the bacterial cells by filtration, preferably by dead-end filtration or microfiltration; and thereby obtaining the protein of interest in the filtrate; wherein the bacterial cells are Bacillus cells. Further, the invention relates to the use of a flocculant for recovering a protein of interest from a bacterial fermentation broth comprising a high degree of lysed cells.

The present invention relates to a method for recovering a protein of interest from a bacterial fermentation broth, wherein the bacterial cells exhibit a high degree of lysis; comprising adding to the fermentation broth comprising the protein of interest at least one flocculant in an amount of 0.5 to 50 g/l of the fermentation broth; wherein the bacterial cells are Bacillus cells; separating the protein of interest from at least the bacterial cells by filtration, preferably by dead-end filtration or cross-flow microfiltration; and obtaining the protein of interest in the filtrate.

In the synthesis step of industrial proteins of interest such as enzymes, the fermentation step, high titers of the protein of interest can be achieved and are also desired. Recent progress in strain and fermentation methodology has led to an increase in density of the cells in fermentation broth, which also results in a further increase in titers of proteins of interest. However, high cell densities may be problematic as unwanted cell lysis may occur. In particular, at the end of fermentation, the broth may contain high levels of lysed cells. Moreover, unwanted lysis of cells may occur when fermentation broths are stored prior to downstream operations for the recovery of the protein of interest, e.g. prior to solid-liquid separation and downstream processing. Cell lysis may be due to unbalanced growth and/or environmental conditions in the fermenter such as shear stress, poor mass and oxygen transfer, toxic waste build-up, metabolic burden from excessive protein expression and internal stress form build-up of protein in the periplasm or storage. Cell lysis typically results in the release of intracellular content such as DNA and protein, including a potential protein of interest, into the fermentation broth. Uncontrolled cell lysis poses a great challenge for downstream operations including clarification/solid-liquid separation and further downstream processing. The resulting cell debris and the presence of extracellular DNA in the fermentation broth are known to hamper downstream operations such as clarification of the fermentation broth and solid-liquid separation, including filtration and centrifugation. However, at present recovery and purification steps must be performed in a certain time window after the end of fermentation while ensuring high overall product recovery yields and product quality. Efficient and simple means offering temporal flexibility for recovering a protein of interest from a fermentation broth comprising a high degree of lysed cells are therefore desired.

US 2005/0176090 A1 discloses a method for purifying an extracellular product of interest from a fungal fermentation broth comprising flocculating the fermentation broth involving a laborious fragmentation/disruption procedure; bacterial fermentation broths with a high degree of lysed cells are not addressed.

WO2014/118220 A1 discloses the use of a flocculant for achieving efficient particle size distribution in a fermentation broth of Escherischia coli cells to realize an efficient clarification method. The method should avoid laborious testing for determining the effective amount of flocculant at various stages of the clarification process, wherein clarification is achieved by centrifugation.

US 2003/129707 A1 discloses a method for producing a protein suspension from a fermentation broth by treating the broth with one or more coagulants and/or one or more flocculants and separating the biomass from the protein by separation, in particular by use of a centrifugation a decanter of a cyclone. US2016/297850 A1 relates to a method for recovering recombinant protein from a mammalian culture broth using cationic polymers, non-ionic polymers and non-ionic surfactants.

US2011/184154 A1 discloses a method for clarification of a cell broth, in particular a mammalian cell broth, with a high density of cells secreting desired biological substances having an overall positive charge in the cell broth by contacting the broth with a particulate anion exchanger.

EP1930419 A1 describes a process for recovering an enzyme from a fermentation broth with a high density of microorganisms using a cationic surfactant.

However, none of the prior art provides efficient and simple means for recovering a protein of interest from a bacterial fermentation broth that exhibit a high degree of lysed cells.

The technical problem underlying the present invention may be seen as the provision of means and methods for complying with the aforementioned needs. It can be solved by the embodiments characterized in the claims and herein below.

The present invention relates to a method for recovering a protein of interest from a bacterial fermentation broth, wherein the bacterial cells in the fermentation broth exhibit a high degree of lysis, comprising the steps of
a) adding to the fermentation broth comprising the protein of interest at least one flocculant in an amount of 0.5 to 50 g/l of the fermentation broth;
b) separating the protein of interest from at least the bacterial cells by filtration, preferably by dead-end filtration or cross-flow microfiltration; and
c) thereby obtaining the protein of interest in the filtrate, wherein the bacterial cells are Bacillus cells.

It is to be understood that as used in the specification and in the claims, “a” or “an” can mean one or more, depending upon the context in which it is used. Thus, for example, reference to “a cell” can mean that at least one cell can be utilized.

Further, it will be understood that the term “at least one” as used herein means that one or more of the items referred to following the term may be used in accordance with the invention. For example, if the term indicates that at least one feed solution shall be used this may be understood as one feed solution or more than one feed solutions, i.e. two, three, four, five or any other number of feed solutions. Depending on the item the term refers to the skilled person understands as to what upper limit the term may refer, if any.

The term “about” as used herein means that with respect to any number recited after said term an interval accuracy exists within in which a technical effect can be achieved. Accordingly, about as referred to herein, preferably, refers to the precise numerical value or a range around said precise numerical value of ±20%, preferably ±15%, more preferably ±10%, or even more preferably ±5%.

The term “comprising” as used herein shall not be understood in a limiting sense. The term rather indicates that more than the actual items referred to may be present, e.g., if it refers to a method comprising certain steps, the presence of further steps shall not be excluded. However, the term also encompasses embodiments where only the items referred to are present, i.e. it has a limiting meaning in the sense of “consisting of”.

Furthermore, the terms “first”, “second”, “third” or “(a)”, “(b)”, “(c)”, “(d)” etc. and the like in the description and in the claims are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. In case the terms “first”, “second”, “third” or “(a)”, “(b)”, “(c)”, “(d)”, “i”, “ii” etc. relate to steps of a method or use or assay, there is no time or time interval coherence between the steps, i.e. the steps may be carried out simultaneously or there may be time intervals of seconds, minutes, hours, days, weeks, months or even years between such steps, unless otherwise indicated in the application as set forth herein above or below.

It is to be understood that this invention is not limited to the particular methodology, protocols, reagents etc. described herein as these may vary. It is also to be understood that the terminology used herein is for the purpose of describing particular embodiments only, and is not intended to limit the scope of the present invention that will be limited only by the appended claims. Unless defined otherwise, all technical and scientific terms used herein have the same meanings as commonly understood by one of ordinary skill in the art.

In the present invention, the inventors have found that a protein of interest can be successfully recovered from bacterial fermentation broths exhibiting a high degree of cell lysis using the method of the invention. In particular, adding a flocculant to the fermentation broth and subsequent filtration can be used to successfully clarify the fermentation broth, for example by separating the protein of interest from at least the bacterial cells (biomass); thereby the protein of interest can be obtained in the filtrate.

Interestingly, the inventors have found that in the method of the invention not only filtration flow rate may be improved during downstream processing but also turbidity of the filtrate may be improved. Advantageously, the method according to the invention offers a simple and efficient way to separate a protein of interest from a bacterial fermentation broth exhibiting a high degree of cell lysis. Moreover, the method according to the invention offers temporal flexibility by allowing storage of the fermentation broth after the end of fermentation, at the time of harvest.

The term “fermentation broth” or “culture broth” as used herein refers to a fermentation medium comprising bacterial cells, which are cultivated to express or produce the protein of interest. In particular, the bacterial cells may be recombinant and/or non-recombinant. More particularly, the recombinant or non-recombinant cells may secrete the protein of interest into the fermentation medium or the protein of interest may leak into the fermentation medium by intended or unintended lysis of the bacterial cells. Hence, the fermentation broth comprising the protein of interest may refer to a fermentation broth comprising bacterial cells that produce the protein of interest, in particular producing the protein of interest during cultivation of the bacterial cells or during the fermentation process. More particularly, the bacterial cells are Bacillus cells.

According to the present invention, the terms “lysis of cells” and “cell lysis” are used interchangeably and are understood as the result of a rupture of cells, in particular bacterial cells. More particularly, cell lysis may refer to a rupture of the cell wall and/or outer membrane of a bacterial cell. In cell lysis, the bacterial cell may lose its cellular integrity; in particular, intracellular content such as DNA, intracellular protein of interest and/or periplasm may be released into the fermentation medium. Hence, cell lysis may therefore be understood as the release of intracellular content into the fermentation broth, in particular in an unintended way such as lysis that occurs automatically without intention and/or human intervention, in particular referring to autolysis or lysis due to storage. In terms of the present invention, cell lysis may be understood to occur automatically, also referred to as autolysis. Cell lysis or autolysis in terms of the present invention may occur during fermentation or after fermentation during storage of the fermentation broth. “Storage of the fermentation broth” may be understood as the period between fermentation and downstream operations, such as solid-liquid separation and/or further downstream processes required for the recovery of the protein of interest. Typically, during storage, the fermentation broth is no longer fermented in the fermenter but resides without further stirring and/or treatment and/or fresh nutrient supply. The duration of the storage may be in the range of several minutes, e.g. 5 min, to 60 h, in particular 10 h to 48 h. Storage may usually at a temperature below room temperature or at room temperature. More specifically, storage may be at 4-25° C., preferably at 4-15, more preferably at 4-10° C. Cell lysis due to storage may generally progress faster with decreasing storage temperatures. Induced lysis, such as lysis occurring due to heating or cooling of the cells, mechanical means including homogenization and freeze-thaw, chemical means including treatment with a lysing agent and osmotic treatment, introduction or expression of lysis inducing proteins, or periplasmic extraction, may preferably not be understood as “cell lysis” in the sense of the present invention. “Lysis of cells” according to the present invention may refer to autolysis of cells, particularly to lysis in bacterial fermentation broths at the end stage of the bacterial life during fermentation as described for example in Newton et el, Biotechn. Prog. 2016, Vol. 32, No. 4, pages 1069-1076; and to lysis occurring during storage of the fermentation broth. Cell lysis and leakage of intracellular content, in particular DNA, into the fermentation medium may increase the viscosity of the fermentation broth. By cell lysis, typically the number of intact cells is reduced. These phenomena may be utilized for determining the degree of bacterial cell lysis.

A person skilled in the art will understand what is meant by a high degree of lysis in a fermentation broth. In accordance with the present invention, a high degree of lysis in particular may refer to a high proportion of bacterial cells in the fermentation broth that have lysed or undergone lysis. In particular, the term “high degree of lysis” may refer to a high proportion of lysed bacterial cells in a fermentation broth producing a protein of interest. More particularly, lysed cells refer to bacterial cells that have lost their cellular integrity, such as membrane integrity and/or that exhibit leakage of intercellular content including DNA and the protein of interest into the fermentation medium.

The degree of bacterial lysis and/or the proportion of lysed bacterial cells in a fermentation broth can be determined by using optical density measurements at 600 nm (OD₆₀₀); shear viscosity determination; scanning electron microscopy (SEM), e.g. for determining the number of intact cells; DNA quantification; online capacitance probes; HPLC quantification of intracellular components; flow cytometry; and/or cytotoxicity assays.

A preferred way of determining cell lysis is determining cell density by optical density measurements at 600 nm (OD₆₀₀). This method is particularly preferred and particularly suitable for determining cell lysis in the case, where the cells have been stored and/or for determining lysis occurring during storage.

Cell density determination by optical density may typically be measured at 600 nm using any type of suitable spectrophotometer, such as spectral colorimeter (Lico 690 from Hach), Ultrospec 500 Pro spectrophotometer (Amersham Biosciences, Amersham, UK), or NanoDrop ND-1000 (Thermofisher Scientific Inc.), or with suitable microplate readers such as Infinite F200 (Tecan, Switzerland). Upon cell lysis cellular density in the fermentation medium decreases and optical density OD₆₀₀ values also significantly decrease (Pasotti et al., J. Biol. Eng. 2011; 5:8). In order to monitor cell density in the fermentation broth, OD₆₀₀ values may be determined at a given time interval, for example every hour up to every 24 hours. Preferably a first measurement is conducted at the end of fermentation, and a second measurement is conducted 24 hours after the end of fermentation; optionally a third measurement may be conducted at 48 hours after the end of fermentation. Preferably, the sample of fermentation broth may be diluted for OD₆₀₀ measurement in order to achieve optimal optical density measurements. More preferably, a sample of fermentation broth may be diluted in water in a ratio (volume fermentation broth/volume water) of 1/500, 1/800, 1/1000 or 1/1200, preferably, 1/1000. According to the present invention, a high degree of lysis may refer to a drop in OD₆₀₀ value of at least 10% between two measurements taken at a suitable time interval, e.g. at the interval between onset of fermentation and 24 hours thereafter or the interval between 24 hours and/or 48 hours after onset. Preferably, a high degree of lysis may refer to a drop in OD₆₀₀ value of at least 20%, at least 30%, or at least 40% between two consecutive measurements, for example two measurements taken within an interval of 24 hours and/or 48 hours between the two of them. More preferably, a high degree of lysis may refer to a drop in OD₆₀₀ value of 20% to 70% between two measurements. More specifically, a drop between 0 hours and 24 hours of between −35% and −45%, preferably, about −40% and/or between 24 hours and 48 hours of between −15% and −25%, preferably about −20% shall be indicative for a high degree of lysis. It will be understood that “about” in this context refers to a deviation of 1 to 2% up or below the precise values referred to.

Moreover, a high degree of lysis may refer to a drop in OD₆₀₀ value of at least 0.1, at least 0.2 or at least 0.3 between two consecutive measurements, for example two measurements taken within an interval of 24 hours between the two of them. Preferably, the absorption values at 600 nm should be in the range between 0.1 and 10. In case the absorption values exceed 10 at 600 nm the sample of the fermentation broth should be diluted in order to ensure proper determination of the cell density.

A further preferred way of determining cell lysis is by measuring g shear viscosity. This method is particularly preferred and particularly suitable when cell lysis occurs already during the fermentation process.

Measuring shear viscosity of the fermentation broth may be done, for example, using rheological measurements and controlling the applied shear rate over a range of 100-1000 s⁻¹. The apparent viscosity (shear viscosity in Pa s) can be determined by recording the viscosity value at a shear rate of 100 s⁻¹ using a rheometer, for example a Kinexus Lab+ rheometer (Malvern Instruments, Malvern, UK) with 50 mm parallel plates at 25° C. and a 300 μm gap size, held at steady state for 10 s.

An increase in viscosity of the fermentation broth of above 20% may be detected upon cell lysis when cell viability is lost and DNA and protein of interest leakage into the fermentation broth occurs (Newton et el, Biotechn. Prog. 2016, Vol. 32, No. 4, pages 1069-1076). In line with the present invention, a high degree of cell lysis in the fermentation broth preferably refers to the condition when an increase in shear viscosity in the fermentation broth of above 20% is detected compared to the viscosity determined at the start of the fermentation. More preferably, an increase in shear viscosity in the fermentation broth of above 25%, of above 30% or of above 35% is referred to as a high degree of cell lysis compared to the viscosity measured at the start of the fermentation.

Thus, in the method according to the invention, a high degree of lysis of the bacterial cells in the fermentation broth corresponds to the degree of lysis that is characterized by (i) a decrease in OD₆₀₀ value of the fermentation broth, preferably a decrease in OD₆₀₀ value of the fermentation broth of at least 10% between two consecutive measurements in samples taken after 24 hours and 48 hours after fermentation when lysis occurs, and/or (ii) an increase in shear viscosity of the fermentation broth, preferably an increase in shear viscosity of above 20% compared to the viscosity determined at the start of the fermentation when lysis occurs during fermentation.

The terms “clarification” and “solid-liquid separation” are used interchangeably herein and are known to the skilled artisan. The terms in particular refer to the removal of the bacterial cells producing the protein of interest from the fermentation broth.

The method according to the present invention is advantageous as it provides a way of protein recovery from cells exhibiting a high degree of unintended lysis such as autolysis or lysis due to storage.

The “start of the fermentation” may refer to the point in time at the induction of the fermentation broth, in the case where the protein of interest is under the control of an inducible or inducer-dependent promoter. In the case where the protein of interest is under the control of a constitutive or self-inducible promoter, the start of fermentation may refer to the point in time, when the inoculum is added to the fermenter in order to start the fermentation process.

However, further methods known to the skilled artisan may also be suitable for determining the degree of lysis such as determining cell density by scanning electron microscopy (SEM); DNA quantification; online capacitance probes; HPLC quantification of intracellular components; flow cytometry; cytotoxicity assays (see e.g. Newton et el, Biotechn. Prog. 2016, Vol. 32, No. 4, pages 1069-1076). In particular, scanning electron microscopy (SEM), and/or DNA quantification may be suitable methods for detecting the degree of lysis of the bacterial cells in the fermentation broth besides determining cell density by optical density measurements at 600 nm (OD₆₀₀) and/or measuring shear viscosity. A combination of the aforementioned methods for determining cell lysis may also be suitable.

As mentioned herein, a further way of determining the degree of cell lysis may be using scanning electron microscopy (SEM). SEM can be used for determining the number of intact cells and disrupted cells in a given volume of a sample. The ratio of disrupted cells to the total number of cells can be used to assess the degree of lysis. For example, the number of disrupted cells can be counted and the number of total cells can be counted. In particular, the following procedure may be performed: the sample of the fermentation broth may be centrifuged and the pellet comprising the cells may be suspended in a suitable primary fixative, such as 2% glutaraldehyde and 0.1 M sodium cacodylate buffer, pH 7.3. The cells may be washed, e.g. in a 0.1 M cacodylate buffer; followed by fixing in a further fixative, such as a 1% osmium tetraoxide in 0.1 M cacodylate buffer. Fixing may be done at 38° C. for 1.5 h. The cells may be washed again, e.g. in 0.1 M cacodylate buffer and washed e.g. with dH₂O, followed by dehydrating in a graded ethanol-water series to 100% ethanol. The samples may then be critical-point dried using CO₂, and mounted on aluminium stubs using sticky carbon taps. The samples may be then coated with a thin layer of a suiable metal or metal alloy such as Au/Pd (2 nm thick) for example by using a Gatan ion beam coater. The samples may be viewed and imaged with a suitable SEM such as a 7401 FEGSEM (Jeol, Mass.).

The degree of disrupted cells [%] may then be calculated as (number of disrupted cells)/(total number of cells in sample)×100%. A high degree of lysis in this case may be regarded as more than 30% of disrupted cells, more than 40%, more than 50%, more than 60%, or more than 70% of disrupted cells in a sample. To receive a reliable estimate, at least three samples should be measured and the calculated degree of disrupted cells should be averaged over the at least three samples.

DNA quantification may be a further way of determining the degree of lysis in accordance with the present invention. DNA in the fermentation broth can be considered to be an indicator of cell lysis. Typically, in the presence of intact cells there should be very little DNA present in the supernatant of a sample of the fermentation broth. Presence of DNA is likely in itself to affect downstream processing as it increases the viscosity of the supernatant and can contribute to loss in effective centrifuge clarification and/or reduced filter flux rates. DNA can be measured using any method known in the art. Typical methods include absorbance measurements at 260 nm/280 nm, and analysis with fluorescence dye tagging. Absorbance measurements at 260 nm/280 nm may be preferred. This method may more preferably additionally comprise absorbance measurements at 260 nm/230 nm for determining organic contamination, e.g. sugars, phenolic compounds, salts. In order to remove contaminating RNA, RNAse treatment may be performed prior to the absorbance measurements. Suitable ways of determining DNA content may include using commercially available kits, such as Quant-iT dsDNA Broad Range Assay kit from Invitrogen, according to manufacturer's instructions. Absorbance measurements may be done using a suitable spectrophotometer such as a NanoDrop ND-1000 (Thermofisher Scientific Inc.). Typically, at an optical path length of 1 cm a double-stranded DNA sample at a concentration of 50 μg/mL will produce an absorbance value (260 nm) of 1. A high degree of cell lysis according to the present invention may refer to a DNA content of 1.5 mg/mL and above, preferably to a DNA content of 1.8 mg/mL or even 2.0 mg/mL and above.

Preferably, the inventive methods further comprise prior to step a) and/or b) the fermentation of the bacterial cells, preferably the fermentation of a bacterial cell producing a protein of interest. More preferably, the bacterial cells in the fermentation broth are cultivated at conditions supporting the production of the protein of interest. Even more preferably, the fermentation broth is cultivated prior to step a) and/or b) until the titer of the protein of interest reaches the desired range. Still even more preferably, when adding the flocculant in step a) the fermentation broth has reached the time of harvest.

In the method according to the present invention, there is preferably no step of lysing the bacterial cells. In particular, there is no step such as heating of the cells, mechanically induced lysis including homogenization and freeze-thaw, lysis by chemicals including treatment with a lysing agent and/or osmotic treatment, introduction or expression of lysis inducing proteins, or periplasmic extraction.

The fermentation process may be of any known set-up, such as a batch process, a fed-batch process or a continuous fermentation process.

A batch fermentation is a process where the growth medium is provided in the fermenter from the start, where the fermenter is inoculated with an intended bacterial cell and the fermentation process is running until a predetermined condition has been reached, typically depletion of the growth medium and the cessation of bacterial growth caused by the depletion.

A fed-batch fermentation is a process where a part of the growth medium is provided from the start of the fermentation process where the inoculum is added, and at a certain time point after the start of the fermentation additional substrate, the feed medium, is fed to the fermenter at a rate that may be predetermined or determined by the conditions in the fermenter, until the maximal volume has been reached. The feed medium may or may not have the same composition as the initial growth medium.

A continuous fermentation is a process where new growth medium is continuously fed to the fermenter and fermentation broth is simultaneously removed from the fermenter at the same rate so the volume in the fermenter is constant. In industrial fermentation processes are typically conducted by first providing a growth medium in a fermenter, inoculating the fermenter with an inoculum comprising a bacterial cell and fermenting under defined conditions such as pH, temperature, oxygen level etc., at a predefined time or until a predefined condition, e.g. titer, oxygen consumption, has been reached.

In a preferred embodiment, a fed-batch fermentation process is performed prior to steps a) and b) of the method of the present invention.

The inoculum is in general a liquid culture of the bacterial cell used for the fermentation prepared in a seed fermenter, a seed fermenter typically having a volume of 5-15% of the main fermenter used for production. The growth medium for the seed fermenter may or may not be the same growth medium as used in the main fermenter.

Thus the inoculum is typically prepared from a vial containing the production strain of the bacterial host cell, where the content of the vial first is inoculated in a small volume and the host cells are grown to a desired density to prepare a first culture of the production strain, where after the first culture the production strain is inoculated in the next of a series of seed fermenters of increasing size, where the volume increases 5-20 fold in each step until a sufficient volume to inoculate the production fermenter has been reached. Such a series of fermenters in increasing size is also known as a seed train (WO2017/068012 A1).

Thus, during the fermentation process, typically a bacterial cell comprising a nucleic acid sequence encoding the protein of interest is inoculated and cultivated in a fermentation medium. Preferably, the bacterial cell is a recombinant bacterial cell.

The term “fermentation medium” refers to a water-based solution containing one or more chemical compounds that can support the growth of cells. Any medium suitable for the culture of the particular bacterial cell may be used. The fermentation medium may be a minimal medium as described before, e.g., in WO 98/37179, or the fermentation medium may be a complex medium comprising complex nitrogen and/or carbon sources, wherein the complex nitrogen source may be partially hydrolyzed as described in WO 2004/003216. Furthermore, the fermentation medium may contain a phosphate and/or carbonate source.

Compounds which may optionally be included in the fermentation medium are chelating agents, such as citric acid, MGDA, NTA, or GLDA, and buffering agents such as mono- and dipotassium phosphate, calcium carbonate, and the like. Buffering agents preferably are added when dealing with processes without an external pH control. In addition, an antifoaming agent may be dosed prior to and/or during the fermentation process. Hence, the fermentation broth may comprise an antifoaming agent.

The present invention may be useful for recovering a protein from any fermentation process in industrial scale, i.e., at least 1,000 liters, more preferably at least 5,000 liters, even more preferably at least 50,000 liters.

A “fermentation process” comprises the cultivation of cells, in particular the bacterial cells, more particularly the Bacillus cells, in a suitable fermentation medium, also referred to as “cultivation medium”. “Cultivation of the cells” or “growth of the cells” is not understood to be limited to an exponential growth phase, but can also include the physiological state of the cells at the beginning of growth after inoculation and during a stationary phase. Typically the cultivation may take place at a cultivation temperature suitable for supporting growth of the cells. More typically, the cultivation temperature may lay within the range of 25° C. to 45° C. Further details are specified elsewhere herein. The fermentation process may usually take place in a fermenter, preferably in a fermenter with a volume scale which is at least 1 m³.

An industrially relevant fermentation process encompasses a fermentation process on a volume scale which is 1-500 m³ with regard to the nominal fermenter size, preferably 5-500 m³, more preferably 10-500 m³, even more preferably 25-500 m³, most preferably 50-500 m³. In other words, an industrially relevant fermentation process encompasses a fermentation process on a volume scale which is at least 1000 L with regard to the nominal fermenter size, preferably at least 5,000 L, more preferably at least 10,000 L, even more preferably at least 25,000 L, still even more preferably at least 50,000 L and most preferably 50,000-500,000 L.

Typically the fermentation process ends or is stopped when a desired titer of the protein of interest is reached. The term “titer of a protein of interest” as used herein is understood as the amount of molecule of interest in g per volume of fermentation broth in liter or in g per kg fermentation broth. The titer of a protein of interest may reach 2 to 100 g molecule/kg fermentation broth at the end of the fermentation process, e.g. at the time of harvest. Preferably, at the time of harvest the titer of a protein of interest reaches the desired range. The desired range may refer to up to 100 g product/kg fermentation broth. More preferably, at the time of harvest the titer of a protein of interest reaches 2 to 100 g product/kg fermentation broth, more preferably the protein of interest reaches 5 to 50 g product/kg fermentation broth at the time of harvest. The term “time of harvest” may particularly refer to the end of the fermentation, more particularly to the point in time when a desirable titer of the protein of interest has been reached, such as 2 to 100 g protein of interest/kg fermentation broth. Even more particularly, the time of harvest refers to the point in time prior to step (a) of the method according to the invention or the point in time prior to storage.

The terms “recovering” or “purifying” may be used interchangeably and are intended to mean “rendering more pure”. They refer to a process in which the protein of interest is separated from other compounds or cells present in the fermentation broth.

“Impurities” in fermentation broth typically include unconverted sugars, residual salts and by-products.

The at least one flocculant according to the present invention is, preferably, a cationic, anionic and/or non-ionic agent, preferably, selected from the group consisting of: organic polymers including cationic, anionic and/or non-ionic polyacrylamides, polyethyleneimine (PEI), poly(diallyldimethylammonium chloride) (pDADMAC), polyamines, natural polymers from microorganisms, and chemical flocculants including soluble Fe or Al compounds. More preferably, organic polymers as flocculants, including cationic and/or anionic polymers comprise polyacrylamides such as Superfloc® C-498 and Superfloc® A-130 (available form Kemira) of different molecular weights, poly(diallyldimethylammonium chloride) (pDADMAC) comprises, preferably, Superfloc® C-592 (from Kemira), polyamines comprise Superfloc® C-521 (available from Kemira), natural polymers from microorganisms comprise, preferably, chitosan, gelatin, alginate or guar gum, chemical flocculants including soluble Fe or Al compounds comprise those disclosed in WO 96/38469, or any mixture thereof, such as Al₂(SO₄)₃, NaAlO₂, K₂Al₂O₄, Al(NO₃)₃, AlCl₃, Al-acetate, Al-formate, Fe₂(SO₄)₃, Fe(III)-formate, Fe(III)-acetate, Fe(II)-formate and Fe(II)-acetate or a polymer aluminum chlorohydrate (e.g., PAX-18 available from Kemira) or Al₂(SO₄)₃. A preferred flocculant in accordance with the present invention is pDADMAC. A commercially available preferred flocculant preparation comprising pDADMAC in accordance with the present invention is Magnafloc® LT7996 (BASF, Solenis).

In the method according to the present invention at least one flocculant is added to the fermentation broth. This means, one or more flocculants may be added to the fermentation broth, including two or more, three or more, or even four or more flocculants. Preferably, the one or more flocculant is added in a concentration in the range of 0.5 to 50 g/L of the fermentation broth, preferably 0.5 to 25 g/L, more preferably 1 to 15 g/L, even more preferably 5 to 10 g/L of the fermentation broth. The flocculant(s) may be added to the fermentation broth before downstream-processing, preferably before the separation of the protein of interest from the biomass.

Moreover, the pH may be adjusted to a range of pH 4.0 to 8.0, preferably to pH 5.0 to 7.0, more preferably to a pH of 6.5. The adjustment may be performed with any suitable acid or base known to the skilled artisan. More preferably, the adjustment can be carried out by adding 25% (v/v) formic acid.

The flocculant may be added under or followed by constant stirring of the broth or mixed using any other means known to the skilled artisan to achieve uniform distribution and thorough mixing.

The fermentation broth may preferably be constantly stirred over a predetermined incubation time after adding the flocculant. The incubation time may vary from 1 to 120 minutes. The incubation time may be 1 minute, 10 minutes, 20 minutes, 30 minutes, 40 minutes, 50 minutes, 60 minutes, 70 minutes, 80 minutes, 90 minutes, 100 minutes, 110 minutes, 110 minutes, or 120 minutes. Preferably, the incubation time is between 10-30 minutes, most preferably, the incubation time is 15 minutes.

In the method according to the invention, the bacterial cell may be a Bacillus cell as specified above comprising an expression construct for a gene encoding an enzyme, in particular a hydrolase, more particularly a protease, even more particularly a serine protease, still even more particularly a subtilisin protease. The gene in the expression construct may be driven by a suitable promoter, preferably an aprE promoter or a derivative thereof, as specified elsewhere herein.

The method according to the invention comprises separating the protein of interest from at least the bacterial cells (biomass) by filtration, preferably by dead-end filtration or cross-flow microfiltration. The filtration may comprise (i) a dead-end filtration step using a Nutsche pressure filter or (ii) cross-flow microfiltration.

Dead-end filtration using a Nutsche pressure filter may comprise high porosity filter aids, in particular perlite filter aids such as Harbolite 900 (Imerys or Lehmann & Voss GmbH & Co. KG), Sil-Kleer 25-M (Silbrico), more particularly further using a diatomite or perlite precoat such as Dicalite BF (Dicalite, Europe) or perlite precoat. Filtration using a Nutsche pressure filter may preferably comprise dilution of the fermentation broth with water using a dilution factor of 1 to 10, preferably a dilution factor of 1 to 3, more preferably a dilution factor of 2. Advantageously, using a Nutsche pressure filter after the addition of a flocculant in the method according to the invention may lead to a decrease in turbidity by a factor of at least 20, at least 30, at least 40, at least 50.

Separation by microfiltration may preferably comprise cross-flow microfiltration using ceramic membranes of a 0.1 to 0.2 μm pore diameter. The microfiltration step can be carried out as diafiltration at constant level, preferably using a diafiltration factor of 1 to 10, preferably a dilution factor of 1 to 5, more preferably a dilution factor of 3. For diafiltration, deionized water may be used. Suitable microfiltration membranes may be made of PESU (polyethersulfone) or based on stabilized cellulose (as commercially available as Hydrosart from Sartorius, Del.

Separating the protein of interest from at least the bacterial cells (biomass) by filtration may refer to removing the biomass or parts thereof in a first fraction while the protein of interest is in the filtrate. In particular, removing at least the biomass by filtration may refer to obtaining the biomass or parts thereof in form of a filter cake or retentate and the protein of interest is obtained in the filtrate or permeate.

Preferably, the obtained filtrate has a turbidity of below 250 NTU, even more preferably of below 200 NTU, still even more preferably of below 150 NTU. Typically, the turbidity is measured at 8° C. Turbidity may be measured using a nephelometer or an instrument capable of determining nephelometric turbidity units such as a spectral colorimeter as the commercially available Lico 690 from Hach.

The term “biomass” refers to the bacterial cells present in the fermentation broth, and fragments or parts of these cells ; in particular the bacterial cells which produce the protein of interest and fragments or parts of these cells present in the fermentation broth. The bacterial cells may be recombinant or non-recombinant. Removing at least the bacterial cells, typically also referred to as “removing the biomass”, or parts thereof, may refer to removing at least 50% of the total amount of biomass initially present in the fermentation broth prior to the filtering step b), preferably at least 60% more preferably at least 70%, even more preferably at least 80%, 85% or 90%. The term “initially present in the fermentation broth prior to the filtering step” may in particular refer to the amount of the biomass such as the bacterial cell density or the total number of bacterial cells at the time of harvest. Methods to determine the amount of biomass in a fermentation broth are known to the person skilled in the art and include for example OD₆₀₀ measurements.

Prior to the separation by filtration, the pH of the fermentation broth may be in the range of 5.2 to 7.2.

In the method according to the invention, subsequent or prior to step c) the protein of interest may be submitted to further downstream processing steps selected from microfiltration, ultrafiltration, diafiltration, dia-ultrafiltration, centrifugation for example with a nozzle separator, extraction, spray-drying, evaporation, precipitation or crystallization. Moreover, it may preferably be further purified by a variety of procedures known in the art including, but not limited to, chromatography (e.g., ion exchange, affinity, hydrophobic, chromatofocusing, and size exclusion), electrophoretic procedures (e.g., preparative isoelectric focusing (IEF), differential solubility (e.g., ammonium sulfate precipitation), or extraction (see, e.g., Protein Purification, J.-C. Janson and Lars Ryden, editors, VCH Publishers, New York, 1989).

The method of the invention may further comprise adding a divalent cation prior to the step (b) of separating the protein of interest from at least the bacterial cells. The divalent cation may be selected from the group consisting of Ca²⁺, Mg²⁺, Ba²⁺, Pb²⁺, Fe²⁺, Zn²⁺, Ni²⁺, Cu²⁺, Mn²⁺, Sr²⁺, Co²⁺ and Be²⁺; preferably the divalent cations is selected from Ca²⁺ or Mg²⁺.

Advantageously, the adding of the divalent cation may further increase the effect of the flocculant and may hence further enhance the membrane flux during downstream processing, increase yield of the protein of interest, and/or improve the turbidity of the filtrate.

The salt of the divalent cation may be the chloride, nitrate, formate, acetate, phosphate or sulfate salt of the divalent cation; preferably the chloride.

Preferably, the salt of the divalent cation is added to the fermentation broth to obtain a final concentration of 0.5 to 10 g/l of the salt of the divalent cation in the broth.

In the method according to the invention, the salt of the divalent cation and the flocculating agent may be added simultaneously or one after the other.

Preferably, the protein of interest is at least partially secreted by the bacterial cells into the fermentation broth, or is expressed intracellularly by the bacterial cells and only unintentionally released from the bacterial cells into the fermentation medium, for example by lysis of the cells.

The bacterial cells may in principle be selected from the group of bacteria consisting of Bacillus, Streptomyces, Escherichia, Buttiauxella and Pseudomonas. In the method of the invention, the bacterial cells are Bacillus cells.

Preferably, the bacterial cells according to the invention refer to a bacterial cell which serves as a host for an expression construct for a gene encoding a protein of interest. Said expression construct may be a naturally occurring expression construct, a recombinantly introduced expression construct or a naturally occurring expression construct which has been genetically modified in the bacterial cell. As mentioned herein, the bacterial cell in the method according to the invention is a Bacillus cell. The Bacillus cell may be a cell from any member of the bacterial genus Bacillus, preferably a host cell of Bacillus licheniformis, Bacillus subtilis, Bacillus alkalophilus, Bacillus amyloliquefaciens, Bacillus brevis, Bacillus circulans, Bacillus clausii, Bacillus coagulans, Bacillus firmus, Bacillus jautus, Bacillus lentus, Bacillus megaterium, Bacillus pumilus, Bacillus stearothermophilus, Bacillus thuringiensis or Bacillus velezensis. More preferably, the Bacillus host cell is a Bacillus licheniformis, Bacillus pumilus, or Bacillus subtilis cell, even more preferred Bacillus licheniformis or Bacillus subtilis cell, most preferably, Bacillus licheniformis host cell. Particular preferably, the Bacillus licheniformis is selected from the group consisting of Bacillus licheniformis as deposited under American Type Culture Collection number ATCC 14580, ATCC 31972, ATCC 53926, ATCC 53757, ATCC 55768, and under DSMZ number (German Collection of Microorganisms and Cell Cultures GmbH) DSM 13, DSM 394, DSM 641, DSM 1913, DSM 11259, and DSM 26543.

Typically, the host cell belongs to the species Bacillus licheniformis, such as a host cell of the Bacillus licheniformis strain as deposited under American Type Culture Collection number ATCC 14580 (which is the same as DSM 13, see Veith et al. “The complete genome sequence of Bacillus licheniformis DSM 13, an organism with great industrial potential.” J. Mol. Microbiol. Biotechnol. (2004) 7:204-211). Alternatively, the host cell may be a host cell of Bacillus licheniformis strain ATCC 53926. Alternatively, the host cell may be a host cell of Bacillus licheniformis strain ATCC 31972. Alternatively, the host cell may be a host cell of Bacillus licheniformis strain ATCC 53757. Alternatively, the host cell may be a host cell of Bacillus licheniformis strain ATCC 53926. Alternatively, the host cell may be a host cell of Bacillus licheniformis strain ATCC 55768. Alternatively, the host cell may be a host cell of Bacillus licheniformis strain DSM 394. Alternatively, the host cell may be a host cell of Bacillus licheniformis strain DSM 641. Alternatively, the host cell may be a host cell of Bacillus licheniformis strain DSM 1913. Alternatively, the host cell may be a host cell of Bacillus licheniformis strain DSM 11259. Alternatively, the host cell may be a host cell of Bacillus licheniformis strain DSM 26543.

Preferably, the Bacillus licheniformis strain is selected from the group consisting of Bacillus licheniformis ATCC 14580, ATCC 31972, ATCC 53757, ATCC 53926, ATCC 55768, DSM 13, DSM 394, DSM 641, DSM 1913, DSM 11259, and DSM 26543.

The term “recombinant” is known to the skilled artisan. In particular, “recombinant” (or transgenic) with regard to a cell or an organism means that the cell or organism contains a heterologous polynucleotide also referred to as “expression construct” which is introduced by man by gene technology and with regard to a polynucleotide includes all those constructions brought about by man by gene technology/recombinant DNA techniques known in the art.

The term “heterologous” (or exogenous or foreign or recombinant or non-native) polypeptide or protein is defined herein as a polypeptide that is not native to the host cell, a polypeptide or protein native to the host cell in which structural modifications, e.g., deletions, substitutions, and/or insertions, have been made by recombinant DNA techniques to alter the native polypeptide or protein, or a polypeptide/protein native to the host cell whose expression is quantitatively altered or whose expression is directed from a genomic location different from the native host cell as a result of manipulation of the DNA of the host cell by recombinant DNA techniques, or whose expression is quantitatively altered as a result of manipulation of the regulatory elements of the polynucleotide by recombinant DNA techniques e.g., a stronger promoter; or a polynucleotide native to the host cell, but integrated not within its natural genetic environment as a result of genetic manipulation by recombinant DNA techniques.

With respect to two or more polynucleotide sequences or two or more amino acid sequences, the term “heterologous” is used to characterize that the two or more polynucleotide sequences or two or more amino acid sequences are naturally not occurring in the specific combination with each other.

The bacterial cell, in particular the Bacillus cell, to be applied in the method of the present invention shall comprise an expression construct for a gene encoding a protein of interest to be expressed by the said cell, also referred to as “host cell”. The term “expression construct” as referred to herein refers to a polynucleotide comprising a nucleic acid sequence encoding the protein of interest operably linked to an expression control sequence, e.g., a promoter. A promoter as referred to herein is a nucleotide sequence located up-stream of a gene on the same strand as the gene that enables transcription of said gene. The promoter is followed by the transcription start site of the gene. The promoter is recognized by an RNA polymerase, typically, together with the required transcription factors, which initiate transcription. A functional fragment or functional variant of a promoter is a nucleotide sequence which is recognizable by RNA polymerase and is capable of initiating transcription. Functional fragments or functional variants of promoters are also encompassed as a promoter in the sense of the pre-sent invention. Promoters may be inducer-dependent promoters the activity of which depend on an activating signal molecule, i.e., the presence of an inducer molecule, or may be inducer-independent promoters, i.e. promoters that do not depend on the presence of an inducer molecule added to the fermentation medium and that are either constitutively active or can be increased in activity regardless of the presence of an inducer molecule that is added to the fermentation medium.

Preferably, the promoter is selected from the group consisting of the promoter sequences of the aprE promoter (a native promoter from the gene encoding the Bacillus subtilisin Carlsberg protease), amyQ promoter from Bacillus amyloliquefaciens, amyL promoter and variants thereof from Bacillus licheniformis (preferably as de-scribed in U.S. Pat. No. 5,698,415), bacteriophage SPO1 promoter, preferably the promoter P4, P5, or P15 (preferably as described in WO2015118126 or in Stewart, C. R., Gaslightwala, I., Hinata, K., Krolikowski, K. A., Needleman, D. S., Peng, A. S., Peterman, M. A., Tobias, A., and Wei, P. 1998, Genes and regulatory sites of the “host-takeover module” in the terminal redundancy of Bacillus subtilis bacteriophage SPO1. Virology 246(2), 329-340), cry-IIIA promoter from Bacillus thuringiensis (preferably as described in WO9425612 or in Agaisse, H. and Lereclus, D. 1994. Structural and functional analysis of the promoter region involved in full expression of the crylllA toxin gene of Bacillus thuringiensis. Mol. Microbiol. 13(1). 97-107.), and combinations thereof, and active fragments or variants thereof. Preferably, the promoter sequences can be combined with 5′-UTR sequences native or heterologous to the host cell, as de-scribed herein. Preferably, the promoter is selected from the group consisting of: an veg promoter, lepA promoter, serA promoter, ymdA promoter, fba promoter, aprE promoter, amyQ promoter, amyL promoter, bacteriophage SPO1 promoter, cryIIIA promoter, combinations thereof, and ac-tive fragments or variants thereof. More preferably, the promoter sequence is selected from the group consisting of aprE promoter, amyL promoter, veg promoter, bacteriophage SPO1 promoter, and cryIIIA promoter, and combinations thereof, or active fragments or variants thereof. More preferably, the promoter is selected from the group consisting of: an aprE promoter, SPO1 promoter, preferably P4, P5, or P15 (preferably as described in WO15118126), tandem promoter comprising the promoter sequences amyl and amyQ (preferably as described in WO9943835), and triple promoter comprising the promoter sequences amyL, amyQ, and cryIIIa (preferably as described in WO2005098016). Most preferably, the promoter is an aprE promoter or a derivative thereof, preferably, an aprE promoter from Bacillus amyloliquefaciens, Bacillus clausii, Bacillus haloduans, Bacillus lentus, Bacillus licheniformis, Bacillus pumilus, Bacillus subtilis, or Bacillus velezensis, more preferably from Bacillus licheniformis, Bacillus pumilus or Bacillus subtilis, most preferably, from Bacillus licheniformis or a derivative thereof. A promoter derivative may refer to a tandem repeat of the promoter sequence or a combination of the promoter with another promoter, preferably with a promoter as listed herein.

It will be understood that the activity of the promoter used in accordance with the method of the present invention, preferably, is not dependent on heat-inducible elements. Accordingly, the promoter to be used as an expression control sequence in accordance of the present invention, preferably, is a temperature-insensitive promoter and/or lacks a heat-inducible element.

Moreover, said expression construct may comprise further elements required for proper termination of translation or elements required for insertion, stabilization, introduction into a host cell or replication of the said expression construct. Such sequences encompass, inter alia, 5′-UTR (also called leader sequence), ribosomal binding site (RBS, Shine-Dalgarno sequence), 3′-UTR, transcription start and stop sites and, depending on the nature of the expression construct, origin of replications, integration sites, and the like. Preferably, the nucleic acid construct and/or the expression vector comprises a 5′-UTR and a RBS. Preferably, the 5′-UTR is selected from the control sequence of a gene selected from the group consisting of aprE, grpE, ctoG, SP82, gsiB, cryIIa and ribG gene.

Yet, the expression construct shall also comprise a nucleic acid sequence encoding a protein of interest. The “protein of interest” as referred to herein refers to any protein, peptide or fragment thereof which is intended to be produced in the bacterial cell. A protein, thus, encompasses polypeptides, peptides, fragments thereof as well as fusion proteins and the like.

Preferably, the Bacillus cells used in the method according to the invention comprise an expression construct comprising a nucleic acid sequence encoding for the protein of interest operably linked to a suitable promoter such as an aprE promoter or a derivative thereof

Preferably, the protein of interest is an enzyme. In a particular embodiment, the enzyme is classified as an oxidoreductase (EC 1), a transferase (EC 2), a hydrolase (EC 3), a lyase (EC 4), an isomerase (EC 5), or a ligase (EC 6) (EC-numbering according to Enzyme Nomenclature, Recommendations (1992) of the Nomenclature Committee of the International Union of Biochemistry and Molecular Biology including its supplements published 1993-1999). In a preferred embodiment, the protein of interest is an enzyme suitable to be used in detergents.

Most preferably, the enzyme is a hydrolase (EC 3), preferably, a glycosidase (EC 3.2) or a peptidase (EC 3.4). Especially preferred enzymes are enzymes selected from the group consisting of an amylase (in particular an alpha-amylase (EC 3.2.1.1)), a cellulase (EC 3.2.1.4), a lactase (EC 3.2.1.108), a mannanase (EC 3.2.1.25), a lipase (EC 3.1.1.3), a phytase (EC 3.1.3.8), a nuclease (EC 3.1.11 to EC 3.1.31), and a protease (EC 3.4); in particular amylase, alpha-amylase, glucoamylase, pullulanase, protease, metalloprotease, peptidase, lipase, cutinase, acyl transferase, cellulase, endoglucanase, glucosidase, cellubiohydrolase, xylanase, xyloglucantransferase, xylosidase, mannanase, phytase, phosphatase, xylose isomerase, glucoase isomerase, lactase, acetolactate decarboxylase, pectinase, pectin methylesterase, polygalacturonidase, lyase, pectate lyase, arabinase, arabinofuranosidase, galactanase, a laccase, peroxidase and an asparaginase; more particularly an enzyme selected from the group consisting of amylase, protease, lipase, lactase, mannanase, phytase, xylanase, phosphatase, glucoamylase, nuclease, and cellulose, even more particularly selected from the group consisting of amylase, mannanase, protease, lactase, preferably the enzyme is an amylase, a mannanase, a lactase, or a protease. Still even more particularly, the enzyme is a protease, still even more particularly a serine protease, most particularly a subtilisin protease.

Further, the fermentation broth, or the fraction thereof, may be diluted before or after the method of the invention is performed. The fermentation broth comprising the protein of interest may be diluted 100-2000% (w/w), preferably 100-1500% (w/w), more preferably 100-1000% (w/w), in particular 200-700% (w/w). The fermentation broth may be diluted with water, preferably deionized water.

Alternatively, the fermentation broth may not be diluted before the method of the invention is performed.

After performing the method according to the invention, the protein of interest may be concentrated by procedures known in the art including, but not limited to, ultrafiltration and evaporation, in particular, thin film evaporation.

Further, the inventive methods may comprise a step d) of preparing a formulation containing the protein of interest.

“Protein formulation” means any non-complex formulation comprising a small number of ingredients, wherein the ingredients serve the purpose of stabilizing the proteins comprised in the protein formulation and/or the stabilization of the protein formulation itself. The term “protein stability” relates to the retention of proteins activity as a function of time during storage or operation. The term “protein formulation stability” relates to the maintenance of physical appearance of the protein formulation during storage or operation as well as the avoidance of bacterial contamination during storage or operation.

The protein formulation can be either solid or liquid. Protein formulations can be obtained by using techniques known in the art. For instance, without being limited thereto, solid enzyme formulations can be obtained by extrusion or granulation. Suitable extrusion and granulation techniques are known in the art and are described for instance in WO 94/19444 A1 and WO 97/43482 A1.

The present invention further relates to the use of a flocculant for recovering a protein of interest from a bacterial fermentation broth comprising a high degree of lysed cells at the time of harvest and/or after storage, wherein the bacterial cells are Bacillus cells. In particular, the use of the flocculant may be refer to the use in the method for recovering a protein of interest from a bacterial fermentation broth as disclosed herein above.

The following shows a list of specific embodiments of the invention:
1. A method for recovering a protein of interest from a bacterial fermentation broth, wherein the bacterial cells in the fermentation broth exhibit a high degree of lysis, comprising the steps of
a) adding to the fermentation broth comprising the protein of interest at least one flocculant in an amount of 0.5 to 50 g/l of the fermentation broth;
b) separating the protein of interest from at least the bacterial cells by filtration, preferably by dead-end filtration or cross-flow microfiltration; and
c) thereby obtaining the protein of interest in the filtrate;
wherein the bacterial cells are Bacillus cells.
2. The method according to embodiment 1, wherein the at least one flocculant is a cationic, anionic and/or non-ionic agent, preferably, selected from the group consisting of: organic polymers including cationic, anionic and/or non-ionic polyacrylamides, polyethyleneimine (PEI), poly(diallyldimethylammonium chloride) (pDADMAC), polyamines, natural polymers from microorganisms, and chemical flocculants including soluble Fe or Al compounds.
3. The method according to any one of the preceding embodiments, wherein the degree of lysis can be measured using optical density measurements at 600 nm (OD₆₀₀); shear viscosity determination; scanning electron microscopy (SEM), e.g. for determining the number of intact cells; DNA quantification; online capacitance probes; HPLC quantification of intracellular components; flow cytometry; and/or cytotoxicity assays.
4. The method according to any one of embodiments 1 to 3, wherein the high degree of lysis of the bacterial cells in the fermentation broth corresponds to the degree of lysis that is characterized by (i) a decrease in OD₆₀₀ value of the fermentation broth, preferably a decrease in OD₆₀₀ value of the fermentation broth of at least 10% between two consecutive measurements in samples taken after 24 hours and 48 hours after fermentation when lysis occurs, and/or (ii) an increase in shear viscosity of the fermentation broth, preferably an increase in shear viscosity of above 20% compared to the viscosity determined at the start of the fermentation when lysis occurs during fermentation.
5. The method according to any one of the preceding embodiments, wherein the method further comprises adding a divalent cation prior to the step (b) of separating the protein of interest from at least the bacterial cells.
6. The method according to the preceding embodiment, wherein the divalent cation is selected from the group consisting of Ca2+, Mg2+, Ba2+, Pb2+, Fe2+, Zn2+, Ni2+, Cu2+, Mn2+, Sr2+, Co2+and Be2+; preferably the divalent cations is selected from Ca2+or Mg2+.
7. The method according to the preceding embodiment, wherein the salt of the divalent cation is the chloride, nitrate, formate, acetate, phosphate or sulfate salt of the divalent cation; preferably the chloride.
8. The method according to any one of the preceding embodiments, wherein the salt of the divalent cation and the flocculating agent are added simultaneously or one after the other.
9. The method according to any one of the preceding embodiments, wherein the at least one flocculant is added in a concentration in the range of 0.5 to 25 g/L, preferably 1 to 15 g/L of the fermentation broth.
10. The method according to any one of the preceding embodiments, wherein the protein of interest is an enzyme.
11. The method according to any one of the preceding embodiments, wherein the protein of interest is selected from the group of enzymes consisting of amylase, alpha-amylase, glucoamylase, pullulanase, protease, metalloprotease, peptidase, lipase, cutinase, acyl transferase, cellulase, endoglucanase, glucosidase, cellubiohydrolase, xylanase, xyloglucantransferase, xylosidase, mannanase, phytase, phosphatase, xylose isomerase, glucoase isomerase, lactase, acetolactate decarboxylase, pectinase, pectin methylesterase, polygalacturonidase, lyase, pectate lyase, arabinase, arabinofuranosidase, galactanase, a laccase, peroxidase and an asparaginase.
12. The method according to any one of the preceding embodiments, wherein the protein of interest is an amylase, a mannanase, a protease or a lactase; preferably a protease.
13. The method according to embodiment 12, wherein the protease is a serine protease, preferably a subtilisin protease.
14. The method according to any one of the preceding embodiments, wherein the protein of interest is driven by an aprE promoter or a derivative thereof.
12. The method according to any one of the preceding embodiments, wherein step a) is performed before step b).
13. The method according to any one of the preceding embodiments, wherein step b) comprises (i) a filtration step using a Nutsche pressure filter or (ii) cross-flow microfiltration.
14. The method according to any one of the preceding embodiments, wherein step b) (i) comprises using high porosity filter aids, in particular perlite filter aids.
15. The method according to any one of the preceding embodiments, wherein subsequent or prior to step c) the protein of interest may be submitted to further downstream processing steps selected from microfiltration ultrafiltration, diafiltration, dia-ultrafiltration, ion-exchange chromatography.
16. The method according to any one of the preceding embodiments, wherein the method further comprises prior to step a) and/or b) the fermentation of the bacterial cells.
17. The method of embodiment 15, wherein the protein of interest is at least partially secreted by the bacterial cells into the fermentation broth.
18. The method according any one of the preceding embodiments, wherein the Bacillus cells are selected from the group consisting of: Bacillus licheniformis, Bacillus subtilis, Bacillus alkalophilus, Bacillus amyloliquefaciens, Bacillus brevis, Bacillus circulans, Bacillus clausii, Bacillus coagulans, Bacillus firmus, Bacillus jautus, Bacillus lentus, Bacillus megaterium, Bacillus pumilus, Bacillus stearothermophilus, Bacillus thuringiensis, and Bacillus velezensis.
19. The method according any one of the preceding embodiments, wherein the Bacillus cells are selected from the group consisting of B. subtilis, B. pumilus and B. licheniformis, preferably the Bacillus cells are cells of B. licheniformis.
20. The method according any one of the preceding embodiments, wherein the Bacillus cells are Bacillus licheniformis cells selected from the group consisting of Bacillus licheniformis as deposited under American Type Culture Collection number ATCC 14580, ATCC 31972, ATCC 53926, ATCC 53757, ATCC 55768, and under DSMZ number (German Collection of Microorganisms and Cell Cultures GmbH) DSM 13, DSM 394, DSM 641, DSM 1913, DSM 11259, and DSM 26543.
21. The method according any one of the preceding embodiments, wherein the Bacillus cells are cells of B. licheniformis, and the protein of interest is a subtilisin protease; preferably the Bacillus cells comprise an expression construct comprising a nucleic acid sequence encoding the subtilisin protease operably linked to an aprE promoter or a derivative thereof.
22. The method according to any one of the preceding embodiments, wherein the salt of the divalent cation is added to the fermentation broth to obtain a final concentration of 0.5 to 10 g/l of the salt of the divalent cation in the broth.
23. The method according to any one of the preceding embodiments, wherein the fermentation broth comprises an antifoaming agent.
24. Use of at least one flocculant for recovering a protein of interest from a bacterial fermentation broth comprising a high degree of lysed cells at the time of harvest and/or after storage, wherein the bacterial cells are Bacillus cells.
25. The use according to embodiment 24, wherein the at least one flocculant is added to the fermentation broth in an amount of 0.5 to 50 g/L of the fermentation broth.
26. The use according to embodiment 24 or 25, wherein the at least one flocculant is a cationic, anionic and/or non-ionic agent, preferably, selected from the group consisting of: organic polymers including cationic, anionic and/or non-ionic polyacrylamides, polyethyleneimine (PEI), poly(diallyldimethylammonium chloride) (pDADMAC), polyamines, natural polymers from microorganisms, and chemical flocculants including soluble Fe or Al compounds.
27. The use according to any of embodiments 24 to 26, wherein the bacterial fermentation broth comprises Bacillus cells producing the protein of interest, preferably Bacillus cells selected from the group consisting of: Bacillus licheniformis, Bacillus subtilis, Bacillus alkalophilus, Bacillus amyloliquefaciens, Bacillus brevis, Bacillus circulans, Bacillus clausii, Bacillus coagulans, Bacillus firmus, Bacillus jautus, Bacillus lentus, Bacillus megaterium, Bacillus pumilus, Bacillus stearothermophilus, Bacillus thuringiensis, and Bacillus velezensis; more preferably the Bacillus cells are Bacillus licheniformis cells.
28. The use according to embodiment 27, wherein the Bacillus cells are Bacillus licheniformis cells selected from the group consisting of Bacillus licheniformis as deposited under American Type Culture Collection number ATCC 14580, ATCC 31972, ATCC 53926, ATCC 53757, ATCC 55768, and under DSMZ number (German Collection of Microorganisms and Cell Cultures GmbH) DSM 13, DSM 394, DSM 641, DSM 1913, DSM 11259, and DSM 26543.
29. The use according to any of embodiments 23 to 28, wherein the protein of interest is an enzyme.
30. The use according to any of embodiments 23 to 29, wherein the method according to any of the preceding embodiments 1 to 23 is performed.

The invention is further illustrated in the following examples which are not intended to be in any way limiting to the scope of the invention as claimed.

### EXAMPLES

The following examples only serve to illustrate the invention. The numerous possible variations that are obvious to a person skilled in the art also fall within the scope of the invention.

Unless otherwise stated the following experiments can be performed by applying standard equipment, methods, chemicals, and biochemicals as used in genetic engineering and fermentative production of chemical compounds by cultivation of microorganisms, in particular bacterial cells. See also Sambrook et al. (Molecular Cloning: A Laboratory Manual. 2nd edition, Cold Spring Harbor Laboratory, Cold Spring Harbor Laboratory Press, Cold Spring Harbor, NY, 1989) and Chmiel et al. (Bioprozesstechnik 1. Einführung in die Bioverfahrenstechnik, Gustav Fischer Verlag, Stuttgart, 1991).

The fermentation broths for the examples below (Examples 1-2) can be obtained by culturing Bacillus licheniformis cells comprising a gene coding for a protein of interest. Bacillus licheniformis cells can be cultivated in a fermentation process using a chemically defined fermentation medium providing the components listed in Table 1 and Table 2. At the end of the fermentation the protein of interest can be solubilized predominantly in the solution phase.

TABLE 1
Macroelements provided during the
course of the fermentation process

| | | Concentration |
|---|---|---|
| Compound | Formula | [g/L initial volume] |
| Citric acid | C₆H₈O₇ | 3.0 |
| Calcium sulfate | CaSO₄ | 0.7 |
| Monopotassium phosphate | KH₂PO₄ | 25 |
| Magnesium sulfate | MgSO₄*7H₂O | 4.8 |
| Sodium hydroxide | NaOH | 4.0 |
| Ammonia | NH₃ | 1.3 |

TABLE 2
Trace elements provided during the
course of the fermentation process

| Trace element | Symbol | Concentration [mM] |
|---|---|---|
| Manganese | Mn | 24 |
| Zinc | Zn | 17 |
| Copper | Cu | 32 |
| Cobalt | Co | 1 |
| Nickel | Ni | 2 |
| Molybdenum | Mo | 0.2 |
| Iron | Fe | 38 |

A solution containing 50% glucose can be used as feed solution. pH can be adjusted during fermentation using ammonia. At the desired product titer the fermentation can be terminated, and the protein of interest can be present in both soluble and crystalline form as confirmed by visual inspection using a microscope.

The fermentation broths for the examples below (Examples 1-3) were obtained by culturing Bacillus licheniformis cells comprising a gene coding for a protease. Bacillus licheniformis cells were cultivated in a fermentation process using a chemically defined fermentation medium. At the end of the fermentation protease is solubilized predominantly in the solution phase.

### Example 1

After harvest the fermentation broth was stored without stirring for 0-48 hours at 22° C. At given time points a sample was drawn and the optical density (OD) at 600 nm was measured using a spectral colorimeter (Lico 690 from Hach). The samples were diluted with water to an OD 600<1 prior to the measurement (Table 3).

TABLE 3
OD600 measurements

| | | OD 600 nm (diluted 1:1000) |
|---|---|---|
| 0 | hrs | 0.303 |
| 24 | hrs | 0.180 |
| 48 | hrs | 0.142 |

The decrease in optical density reflects an increase in cell lysis.

### Example 2

Fermentation broth was stored for 48 hrs after harvest at 22° C. as in Example 1. The biomass was separated using a dead-end filtration pressure nutsche unit on 200 mL scale using a high porosity perlite filter aid and a diatomite precoat:
Body feed: 40 g/kg Harbolite 900
Precoat: 0.05 g/cm² Dicalite BF

Filtrations were performed at 10° C. Before filtration the fermentation broth was adjusted to pH 6.5 and subsequently diluted with flocculation solution (positive control) or deionized water (negative control) using the same dilution factor for all runs.

Run 1: The diluted fermentation broth was used without flocculant addition (negative control).

Run 2: The fermentation broth was pretreated using pDADMAC flocculation solution before the filtration step: 40 g Magnafloc® LT7996 (˜20% w/v pDADMAC content) per 1 L of fermentation broth was diluted to the respective volume using deionized water to make up the flocculation solution. This solution was subsequently mixed with the fermentation broth under constant stirring. After 15 min the pretreated broth was subjected to dead-end filtration, as in run 1.

TABLE 4
Turbidity measurements

| | Run 1 | Run 2 |
|---|---|---|
| Turbidity after filtration | over range | 125 |
| (measured at 8° C.) [NTU] | | |

As shown in Table 4 the turbidity of the filtrates was much lower when the fermentation broth was pretreated with flocculant (run 2) than without flocculant pretreatment.

The turbidity was measured using a Hach turbidity meter and all measurements were performed at a temperature of about 8° C. to account for background turbidity resulting from antifoaming agents.

### Example 3

The two filtrates of example 2 (run 1 and run 2) were subsequently filtered using a depth filter (Seitz K250 from Pall) precoated with 0.1 g/cm² Dicalite BF at 10° C. for polish filtration. The same pressure nutsche apparatus as in in the first filtration (example 2) was used for these filtrations.

Run 3: Filtrate of run 1 (negative control)

Run 4: Filtrate of run 2

The filtrate volume was recorded as a function of time and the turbidity was measured using the same instrument as before.

TABLE 5
Turbidity measurements after K250 filtration

| | Run 3 | Run 4 |
|---|---|---|
| Turbidity after K250 filtration | 13 | 4 |
| (measured at 8° C.) [NTU] | | |
| Filtrate volume after 100 s [mL] | 6.4 | 149.2 |

The turbidity of the filtrate in run 4 which originated from flocculated fermentation broth in the first step (example 2) was lower compared to run 3 (negative control)(Table 5). Additionally, the flow through the filter was markedly better in the case of run 4 (flocculated material) compared to run 3 (negative control).

### Example 4

The biomass can be separated using a cross-flow microfiltration unit on 3 L scale equipped with ceramic membranes 0.1-0.2 μm pore diameter. The microfiltration step can be carried out as diafiltration at constant level (diafiltration factor of DF=4). For diafiltration, deionized water can be used.

Run 5: The fermentation broth will be used without pretreatment (negative control).

Run 6: A suitable flocculant will be added before the microfiltration step: e.g. 40 g Magnafloc® LT7996 (˜20% w/v pDADMAC content) can be added under constant stirring per 1 L of fermentation broth. After 15 min the pretreated broth will be subjected to microfiltration, as in run 5. In both runs the initial pH of the treated and untreated fermentation broth before microfiltration will be in a range of pH 6.2-7.2.

Adding a suitable flocculant to the fermentation broth is expected to improve the performance of the microfiltration operation in terms of higher yield and better fluxes across the membrane. Increases in yield of up to 10% are expected and the increase in flux across the membrane is expected to be in the range of 20 to 400%.

### Example 5

The biomass can be separated using a dead-end filtration pressure nutsche unit on 200 mL scale using a high porosity perlite filter aid and a diatomite precoat: for example a Body feed: 40 g/kg Harbolite 900 and Precoat: 0.05 g/cm² Dicalite BF. Before filtration the fermentation broth can be diluted with deionized water for both runs using the same dilution factor.

Run 7: The diluted fermentation broth will be used without pretreatment (negative control).

Run 8: A suitable flocculant, e.g. will be added to the diluted fermentation broth before the microfiltration step: 40 g Magnafloc® LT7996 (˜20% w/v pDADMAC content) can be added under constant stirring per 1 L of fermentation broth to obtain a pretreated broth. The stirring will be continued for a suitable time, e.g. 15 min. The pretreated broth will be subjected to dead-end filtration, as in run 7. The results are expected to show that the pretreated broth exhibits significantly lower turbidity after the run, e.g. a decrease by a factor of 20, 30, 40 or more.

1. A method for recovering a protein of interest from a bacterial fermentation broth, wherein bacterial cells in the fermentation broth exhibit a high degree of lysis, comprising
a) adding to the fermentation broth comprising the protein of interest at least one flocculant in an amount of 0.5 to 50 g/L of the fermentation broth;
b) separating the protein of interest from at least the bacterial cells by filtration; and
c) thereby obtaining the protein of interest in the filtrate;
wherein the bacterial cells are Bacillus cells.

2. The method according to claim 1, wherein the at least one flocculant is a cationic, anionic and/or non-ionic agent selected from the group consisting of: cationic, anionic, and/or non-ionic polyacrylamides, polyethyleneimine (PEI), poly(diallyldimethylammonium chloride) (pDADMAC), polyamines, natural polymers from microorganisms, and soluble Fe or Al compounds.

3. The method according to claim 1, wherein the degree of lysis is measured using optical density measurements at 600 nm (OD₆₀₀); shear viscosity determination; scanning electron microscopy (SEM); DNA quantification; online capacitance probes; HPLC quantification of intracellular components; flow cytometry; and/or cytotoxicity assays.

4. The method according to claim 1, wherein the high degree of lysis of the bacterial cells in the fermentation broth corresponds to the degree of lysis that is characterized by (i) a decrease in OD₆₀₀ value of the fermentation broth and/or (ii) an increase in shear viscosity of the fermentation broth.

5. The method according to claim 1, wherein the method further comprises adding a divalent cation prior to step (b) of separating the protein of interest from at least the bacterial cells.

6. The method according to claim 1, wherein the at least one flocculant is added in a concentration in a range of 0.5 to 25 g/L of the fermentation broth.

7. The method according to claim 1, wherein the protein of interest is an enzyme.

8. The method according to claim 1, wherein the protein of interest is selected from the group of enzymes consisting of amylase, alpha-amylase, glucoamylase, pullulanase, protease, metalloprotease, peptidase, lipase, cutinase, acyl transferase, cellulase, endoglucanase, glucosidase, cellubiohydrolase, xylanase, xyloglucantransferase, xylosidase, mannanase, phytase, phosphatase, xylose isomerase, glucoase isomerase, lactase, acetolactate decarboxylase, pectinase, pectin methylesterase, polygalacturonidase, lyase, pectate lyase, arabinase, arabinofuranosidase, galactanase, a laccase, peroxidase, and an asparaginase.

9. The method according to claim 1, wherein step a) is performed before step b).

10. The method according to claim 1, wherein step b) comprises (i) a filtration step using a Nutsche pressure filter or (ii) cross-flow microfiltration.

11. The method according to claim 1, wherein step b) (i) comprises using high porosity filter aids.

12. The method according to claim 1, wherein subsequent or prior to step c), the protein of interest is submitted to further downstream processing steps selected from microfiltration, ultrafiltration, diafiltration, dia-ultrafiltration, and ion-exchange-chromatography.

13. The method according to claim 1, wherein the method further comprises prior to step a) and/or b) a fermentation of the bacterial cells.

14. The method according to claim 1, wherein the Bacillus cells are selected from the group consisting of: Bacillus licheniformis, Bacillus subtilis, Bacillus alkalophilus, Bacillus amyloliquefaciens, Bacillus brevis, Bacillus circulans, Bacillus clausii, Bacillus coagulans, Bacillus firmus, Bacillus jautus, Bacillus lentus, Bacillus mega-terium, Bacillus pumilus, Bacillus stearothermophilus, Bacillus thuringiensis, and Bacillus velezensis.

15. (canceled)

16. The method of claim 1 wherein separating in step b) is by dead-end filtration or microfiltration.
